(19) United States
(12) Patent Application Publication
(10) Pub. No.: US 2022/0356452 A1
(43) Pub. Date: Nov. 10, 2022

(54) INDIRECT THREE-DIMENSIONAL CO-CULTURE OF DORMANT TUMOR CELLS AND USES THEREOF

(71) Applicants: April Kloxin, Landenberg, PA (US); Lina Pradhan, Newark, DE (US); Robert Sikes, Elkton, MD (US); Kenneth van Golen, Newark, DE (US); DeVonte Moore, Newark, DE (US)

(72) Inventors: April Kloxin, Landenberg, PA (US); Lina Pradhan, Newark, DE (US); Robert Sikes, Elkton, MD (US); Kenneth van Golen, Newark, DE (US); DeVonte Moore, Newark, DE (US)

(73) Assignee: University of Delaware, Newark, DE (US)

(21) Appl. No.: 17/764,593

(22) PCT Filed: Sep. 30, 2020
(86) PCT No.: PCT/US2020/053376

§ 371 Date: Mar. 29, 2022

Related U.S. Application Data
(60) Provisional application No. 62/907,876, filed on Sep. 30, 2019.

(51) Int. Cl. C12N 5/09; C12N 5/077

(52) U.S. Cl. CPC C12N 5/0693; C12N 5/0669; C12N 2502/1311; C12N 2513/00; C12N 2500/00

(57) ABSTRACT

The present invention relates to an indirect three-dimensional co-culture. The indirect co-culture may comprise bone marrow niche cells, tumor cells and a culture medium. The bone marrow niche cells and the tumor cells may be incubated in the culture medium without direct contact between the bone marrow niche cells and the tumor cells. The tumor cells may be dormant or reactivated. Also provided are a method for preparing the indirect co-culture and a method for screening for an agent capable of inhibiting reactivation of dormant tumor cells or promoting dormancy of proliferating tumor cells.

### CROSS-REFERENCE TO RELATED APPLICATION

This application claims priority to U.S. Provisional Application No. 62/907,876, filed Sep. 30, 2019, and the contents of which are incorporated herein by reference in their entireties for all purposes.

### FIELD OF THE INVENTION

The invention relates to an indirect three-dimensional co-culture of dormant tumor cells and preparation and uses thereof.

### BACKGROUND OF THE INVENTION

Estrogen receptor (ER)-positive (ER+) breast cancer is accountable for late recurrence after a diagnosis of five (5) years and where the disseminating cancer cells most often colonize into the distant bone marrow site. The disseminated ER+ breast cancer cells (BCCs) adapt to their new complex microenvironment with bone marrow niche cells and remain in a long-period of dormancy, a survival state, before metastatic growth or remain active, partly in response to the tumor-associated bone marrow (BM) cells. These types of processes in dormancy and reactivation are important in other types of cancers, including different breast cancer subtypes, and other cancer types such as prostate and skin cancer, as well as other metastatic niches such as the lung.

ER+ breast cancers follow multiple processes of distance metastasis colonization: 1) circulating tumor cells (CTCs) most often colonizes into the bone marrow site and contact in the local microenvironment or niche; 2) after arriving into the bone marrow site, disseminated ER+ BCCs accustom to their new complex microenvironment with several types of bone marrow niche cells that secrete soluble factors, and the BCCs enter into a long-period dormant and survival state before colonization/metastasis or are activate by the tumor-associated bone marrow cells; 3) the dormant BCCs reactivate from a dormant to proliferative state over long times to develop a micrometastases, where the mechanism for this complex process remains not well understood and hard to study; and 4) after switching from dormant to proliferative state, cells grow uncontrollably and modify the bone microenvironment as the metastasis.

All the progress needs to be understood that how, what, and when disseminated tumor cells (DTCs) become in a dormant, activation, or reactivation states in the bone marrow site, for example, how tumor cells change the bone marrow microenvironment and how this microenvironment can control the growth of tumor cells, what key cues play a role in dormancy, activation, and reactivation of tumor cells, and when these dormant cells wakeup. There are limited information and knowledge of early colonization, dormancy and survival, and the reactivation of DTCs. Researchers are trying to develop new technologies that can facilitate the study of dormancy and reactivation of tumor cells and provide insights into mechanism for targeting them and the development of new treatment strategies.

Insights into the mechanism of the long-period survival of dormant BCCs at the distant site remain unclear and needed for the design of improved treatment strategies. There is a need for a well-defined, indirect co-culture 3D model to understand the complexity of tissue environment effects on cancer dormancy and reactivation and allow the evaluation of different types of therapeutics and therapeutic dosing strategies.

### SUMMARY OF THE INVENTION

The present invention relates to an indirect three-dimensional co-culture of tumor cells and metastatic niche cells in the same culture medium without physical contact with each other. The inventors have surprisingly discovered such an indirect co-culture of breast cancer cells (BCCs) and bone marrow niche cells is useful for mimicking a metastatic niche microenvironment for regulating dormancy, activation and reactivation of the BCCs.

An indirect co-culture is provided. The indirect co-culture comprises bone marrow niche cells, tumor cells and a culture medium. The bone marrow niche cells and the tumor cells are incubated in the culture medium without direct contact between the bone marrow niche cells and the tumor cells. At least 65% of the tumor cells are viable for at least 15 days.

According to the indirect co-culture, at least 80% of the tumor cells may be dormant. The bone marrow niche cells may be human bone lining osteoblast cells (hFOBs). The culture medium may comprise a soluble dormancy factor in an amount effective for promoting dormancy of the tumor cells. The soluble dormancy factor may be selected from the group consisting of cytokines, chemokines, enzymes, extracellular vesicles, growth factors, and combinations thereof. The soluble dormancy factor may be secreted by the bone marrow niche cells.

According to the indirect co-culture, at least 80% of the tumor cells may be reactivated. The bone marrow niche cells may be human mesenchymal stem cells (hMSCs). The culture medium may comprise a soluble reactivation factor in an amount effective for promoting reactivation of the tumor cells. The soluble reactivation factor may be selected from the group consisting of cytokines, chemokines, enzymes, extracellular vesicles, growth factors, and combinations thereof. The soluble reactivation factor may be secreted by the bone marrow niche cells.

According to the indirect co-culture, the culture medium may comprise an autophagy inhibitor. The autophagy inhibitor may be hydroxychloroquine (HCQ).

According to the indirect co-culture, the tumor cells may be selected from the group consisting of breast cancer cells, bone cancer cells, brain cancer cells, lung cancer cells, prostate cancer cells, and skin cancer cells. The tumor cells may be estrogen receptor-positive (ER+) breast cancer cells. The tumor cells may be obtained from a patient having an associated primary tumor. The tumor cells may be disseminated tumor cells (DTCs). The tumor cells may be selected from the group consisting of T47D, ZR-75-1, and BT474.

According to the indirect co-culture, the bone marrow cells may be in a 3D matrix and/or the tumor cells may be in a 3D matrix.

A method for preparing an indirect co-culture is also provided. The preparation method comprises incubating bone marrow niche cells and tumor cells in a culture medium without direct contact between the bone marrow niche cells and the tumor cells. At least 65% of the tumor cells are viable for at least 15 days.

According to the preparation method, at least 80% of the tumor cells may be dormant. The bone marrow niche cells may be human bone lining osteoblast cells (hFOBs). The culture medium may comprise a soluble dormancy factor in an amount effective for promoting dormancy of the tumor cells. The soluble dormancy factor may be selected from the group consisting of cytokines, chemokines, enzymes, extracellular vesicles, growth factors, and combinations thereof. The soluble dormancy factor may be secreted by the bone marrow niche cells.

According to the preparation method, at least 80% of the tumor cells may be reactivated. The bone marrow niche cells may be human mesenchymal stem cells (hMSCs). The culture medium may comprise a soluble reactivation factor in an amount effective for promoting reactivation of the tumor cells. The soluble reactivation factor may be selected from the group consisting of cytokines, chemokines, enzymes, extracellular vesicles, growth factors, and combinations thereof. The soluble reactivation factor may be secreted by the bone marrow niche cells.

According to the preparation method, the culture medium may comprise an autophagy inhibitor. The autophagy inhibitor may be hydroxychloroquine.

According to the preparation method, the tumor cells may be selected from the group consisting of breast cancer cells, bone cancer cells, brain cancer cells, lung cancer cells, prostate cancer cells, and skin cancer cells. The tumor cells may be estrogen receptor-positive (ER+) breast cancer cells. The tumor cells may be obtained from a patient having an associated primary tumor. The tumor cells may be disseminated tumor cells (DTCs). The tumor cells may be selected from the group consisting of T47D, ZR-75-1, and BT474.

According to the preparation method, the bone marrow cells may be in a 3D matrix and/or the tumor cells may be in a 3D matrix.

For each preparation method of the present invention, an indirect co-culture prepared according to the method is provided.

A method for screening for an agent capable of inhibiting reactivation of dormant tumor cells is provided. The screening method comprises incubating bone marrow niche cells and dormant tumor cells in a culture medium without direct contact between the bone marrow niche cells and the dormant tumor cells. At least 80% of the dormant tumor cells become reactivated. The screening method further comprises adding a test agent into the culture medium, and determining the percentage of the dormant tumor cells that become reactivated before and after the test agent is added. A decrease in the percentage of the reactivated tumor cells after the addition of the test agent indicates that the test agent inhibits reactivation of the dormant tumor cells. The bone marrow niche cells may be human bone lining osteoblast cells (hFOBs). The screening method may further comprise inhibiting autophagy.

A method for screening for an agent capable of promoting dormancy of proliferating tumor cells is provided. The screening method comprises incubating bone marrow niche cells and proliferating tumor cells in a culture medium without direct contact between the bone marrow niche cells and the proliferating tumor cells. At least 80% of the proliferating tumor cells become dormant. The screening method further comprises adding a test agent into the culture medium, and determining the percentage of the proliferating tumor cells that become dormant before and after the test agent is added. An increase in the percentage of the dormant tumor cells after the addition of the test agent indicates that the test agent promotes dormancy of the tumor cells. The bone marrow niche cells may be human mesenchymal stem cells (hMSCs). The screening method may further comprise inhibiting autophagy.

### BRIEF DESCRIPTION OF THE DRAWINGS

FIG. 1 illustrates bio-inspired bone marrow metastatic niche microenvironment: in vitro well-defined 3D co-culture model for cancer dormancy and reactivation.

FIGS. 2a-e show viability and metabolic activity of ER+ breast cancer cells in 3D direct and indirect co-culture with human bone lining osteoblast cells (hFOBs) (dormancy-promoting microenvironment) or human mesenchymal stem cells (hMSCs) (growth-promoting microenvironment). a, Approach for direct and indirect 3D co-culture of bone marrow (BM) niche cells (hMSC or hFOB) with BCCs. b & c, ER+T47D or ER+ HER2+BT474 alone (mono-culture growth control), in direct co-culture (+), or in indirect co-culture (#)). Quantitative analysis of viable cells. d & e, Metabolic activity over time.

FIG. 3a-e show probing effects of bone marrow niche cell secreted factors with indirect 3D co-cultures. a & b, Quantification of proliferative (EdU^{+ve}) T47D & BT474 cells alone or in indirect co-culture with different BM niche cells (hFOB or hMSC). c, Approach for assessing response to change in microenvironment with transwell insert exchange. d, Proliferation assessment with insert exchange using EdU proliferation assay, here with quantitative analysis of EdU^{+ve}T47D cells before and after insert exchange. e, Quantitative analysis of metabolic activity of T47D cells before and after insert exchange.

FIGS. 4a-e show induction of autophagy in BCCs upon entering a dormant state in 3D indirect co-culture over time. a & b, Images of immunofluorescent staining of autophagic protein (LC3B), cytoplasm (Phalloidin), and DNA (Hoechst) of T47D and BT474 cells in 3D culture were quantitatively analyzed of cells positive for LC3B^{+ve} puncta. Shown here, quantitative analysis of BCC with LC3B^{+ve} puncta cultured in three dimensions in mono-culture or in indirect co-culture with different BM niche cells (hFOB or hMSC). c, Schematic representation of switching microenvironment by transwell insert exchange between two different co-cultured conditions. d & e, Quantitative analysis of cells positive for LC3B^{+ve} puncta before and after insert exchange.

FIGS. 5a-b show assessment of cytokines secreted by cells for probing regulators of dormancy vs. persistent activation and reactivation for a, T47D and b, BT474 cells in mono-culture or indirect co-culture.

FIGS. 6a-f show probing effects of identified cytokines on BCCs viability and growth. a, Approach for probing importance of cytokine binding on induction of dormancy and autophagy. b & c, Quantitative analysis of viable cells and metabolic activity over time in three dimensions in mono-culture or in indirect co-culture with different BM niche cells (hFOB) after blocking of cytokine receptors for T47D. d, Schematic representation of probing effects of cytokines on the induction of dormancy and autophagy in 3D cultures. e & f, Quantitative analysis of viable cells and metabolic activity over time in three dimensions with treatment of T47D cells with TNFa and MCP1 cytokines in comparison to controls (mono-culture or in indirect co-culture with different BM niche cells (hFOB)).

FIGS. 7a-e show probing effects of cytokines on induction of dormancy and autophagy in dormancy-inducing hFOB co-culture. a & b, Quantification of proliferative EdU^{+ve} and autophagic LC3^{+ve} T47D cells over time in dormancy-inducing hFOB co-culture with and without treatment with anti-TNFR1 and anti-CCR2 in comparison to 3D mono-culture control. c & d, Quantification of proliferative EdU^{+ve} and autophagic LC3^{+ve} T47D cells over time with treatment of TNFa and MCP1 cytokines in comparison to controls (mono-culture or in indirect co-culture with different BM niche cells (hFOB)). e, A bar graph of quantification analysis on EdU^{+ve} and LC3B^{+ve}T47D cells at day 15 time points from c & d.

FIGS. 8a-b show dose-dependent response to inhibitor of autophagy in indirect co-culture model. a, Overview of study for probing effects of inhibition of autophagy over time. b, Quantitative analysis of LC3B^{+ve} puncta cells in T47D #hFOB co-culture conditions with and without treatment.

### DETAILED DESCRIPTION OF THE INVENTION

The present invention relates to indirect co-culture of tumor cells and bone marrow niche cells mimicking a bone marrow microenvironment that regulates dormancy, activation, and reactivation of the tumor cells. The inventors have surprisingly discovered a well-defined, synthetic 3D indirect co-culture model mimicking both cell-matrix and cell-cell interactions in the bone marrow microenvironment and for unique insights into secreted factors that regulate dormancy, activation, and reactivation of ER+ breast cancer cells. This system affords opportunities for mimicking other metastatic niches by tailoring the properties of the synthetic matrix and utilizing different niche cells and examining responses of different cancer cell types.

To develop the indirect co-culture, the inventors have developed and applied various techniques. Intracardiac injection of ER+ breast cancer cells allows developing a bone metastatic dormancy model for studying the mechanism of survivability of dormant cells in the bone marrow microenvironment for comparison to the in vitro model system. A bioactive and cell-adhesive bioinspired synthetic matrix has been developed to afford independent tuning of modulus and mimic bone marrow tissue. The 3D indirect co-culture synthetic model mimics both cell-matrix and cell-cell interactions in the bone marrow microenvironment and provides unique insights into secreting factors by bone cells that regulate dormancy, activation, and reactivation of ER+ breast cancers. Bone marrow niche cells especially mesenchymal stem cells (hMSC) and osteoblast (hFOB) cells play a key role for insights into the mechanism of survival and reactivation of dormant ER+ breast cancer cells. The hMSC influence proliferation to the dormant ER+ BCCs where hFOB cells induce dormancy that followed specific cytokines pathways. This indirect co-culture model demonstrates that the osteoblast cell-derived cytokines, especially TNFa and MCP1, induce dormancy of ER+ BCCs. The dormant ER+ BCCs maintain their survivability through autophagy mechanism in the bone marrow microenvironment. Targeting of dormant ER+ BCCs can switch either in proliferation or in remaining dormancy. Inhibition of autophagy of dormant cells using hydroxychloroquine (HCQ as a model drug) treatment can switch in metastasis or apoptosis. Targeting the specific cytokine pathway either can switch in the proliferation of dormant ER+ BCCs or can maintain the dormancy.

The inventors have developed an in vitro dormancy model of ER+ breast cancer and investigated that autophagy is a key mechanism of long-period survival of dormant ER+ breast cancer cells and where this disseminated dormant cells localized within the bone marrow in close proximity to bone lining cells at the bone surface. To probe dynamic cell-cell indirect interactions regulating dormancy within this niche, the inventors have established and utilized a well-defined dynamic indirect co-culture model of ER+ breast cancer cells with different metastatic potentials (T47D, ZR-75-1, BT474) with key bone marrow niche cells, specifically human mesenchymal stem cells (hMSC) and bone lining human osteoblast cells (hFOB). The inventors have demonstrated that hMSCs influence the growth of the ER+ breast cancer cells, whereas hFOB cells promote dormancy. The inventors have discovered that osteoblastic niche releases soluble factors, for example, TNFa and MCP1, which can suppress the growth and induce survival in dormancy. This suppression of growth of BCCs is reversible, either by exchanging the microenvironment or by targeting to inhibit the self-renewal survival mechanism autophagy. Targeted cytokine mediated signaling to inactivate autophagy in the dormant BCCs leads to a switch from dormancy into proliferation. The osteoblastic niche is essential in controlling ER+ BCCs dormancy and creates the potential for targeting autophagy mechanism to prevent a late recurrence. The unexpected results affirm that well defined indirect co-culture model with osteoblast cells established a dormant niche, whereas mesenchymal cells promote growth and activation of the dormant cells.

The term “indirect co-culture” used herein refers to an in vitro culture in which bone marrow niche cells and tumor cells are incubated in the same culture medium without physical contact with each other. The bone marrow niche cells comprise one or more types of cells. The tumor cells may comprise one or more types of cells. The bone marrow niche cells and the tumor cells do not comprise the same type of cells.

The term “bone marrow niche cells” used herein refers to cells that are obtained from or derived from cells obtained from bone marrow and capable of inducing a biological change in the tumor cells in an indirect co-culture. Examples of the bone marrow niche cells include human bone lining osteoblast cells (hFOBs) (also denoted as human bone lining osteoblast (hFOB)), human mesenchymal stem cells (hMSCs) (also denoted as human mesenchymal stem cells (hMSC)), human vascular endothelial cells, human hematopoietic stem cells or a combination thereof. In the indirect co-culture, the hFOBs may create a dormancy-promoting microenvironment and induce the tumor cells to maintain a dormant state or enter a dormant state from an activation or reactivation state while the hMSCs may create a growth-promoting microenvironment and induce the tumor cells to maintain an activation or reactivation state or enter a reactivation state from a dormant state.

The term “tumor cells” used herein refers to cells that are obtained from or derived from cells obtained from a tumor in a subject and are in a dormant state and capable of being reactivated, or activated or reactivated and capable of entering a dormant state. The tumor may be in breast, bone, brain, lung, prostate or skin. The tumor may be a primary tumor. The tumor cells may be disseminated tumor cells (DTCs). Exemplary tumor cells include breast cancer cells, bone cancer cells, brain cancer cells, lung cancer cells, prostate cancer cells, and skin cancer cells. In one embodiment, the tumor cells are breast cancer cells (BCCs). The BCCs may be estrogen receptor-positive (ER+). The ER+ BCCs may be T47D, ZR-75-1, BT474 or a combination thereof.

The term “disseminated tumor cells (DTCs)” used herein refers to tumor cells that have left a primary tumor in a subject and disseminate to a secondary site, for example, a distant organ in the body (e.g., bone, lung, liver, brain, or other organ) of the subject. The secondary site may be represented by metastatic niche cells (e.g., bone morrow niche cells, bone niche cells, lung niche cells or liver niche cells) and a synthetic matrix within the indirect co-culture model of the present invention. The DTCs may be dormant, activated or reactivated at the secondary site.

The term “subject” used herein refers to a mammal, for example, a human. The subject may be a patient having an associated primary tumor. The associated primary tumor may be in the breast, bone, brain, lung, prostate or skin of the subject. In one embodiment, the subject has a breast tumor and estrogen receptor-positive (ER+) tumor cells are from the breast tumor.

The term “biological change” used herein refers to a change in morphology, viability, proliferation, expression of biomarkers, or other biological properties of cells. The biological change of tumor cells may be a transition between two different states, for example, a dormant, activation, or reactivation state.

The terms “grow” and “proliferate” are used interchangeably herein and refer to viable cells exhibiting an increasing cell number for a predetermined period of time. Viable cells having an increasing cell number rate of at least 1, 5, 10, 25, 50 or 100 times every 24 hours for a predetermined period of time are deemed growing or proliferating. Viable cells not having an increasing cell number rate of at least 1 or 5 times every 24 hours for a predetermined period of time are deemed not growing or proliferating. Cells positive for markers of proliferation may be deemed growing or proliferating. For example, EdU incorporation into DNA that occurs during S-phase of a cell cycle or Ki-67 expression may be evidence of growing or proliferating cells.

The term “dormant”, “dormant state” or “dormancy” used herein refers to viable tumor cells that do not grow or proliferate but retain the ability to grow upon stimulation at a later time.

The term “activated”, “activation state” or “activation” used herein refers to viable cells that are growing or proliferating for a predetermined period of time. The proliferating tumor cells may be reactivated tumor cells.

The term “reactivated”, “reactivation state” or “reactivation” used herein refers to viable tumor cells that are growing or proliferating for a predetermined period of time after being dormant or in a dormant state.

The term “dormancy factor” used herein refers to an agent soluble in a culture medium that maintains dormancy of tumor cells in the culture medium or promotes transition of tumor cells in the culture medium from an activation or reactivation state to a dormant state. The agent may be a chemical compound, a biological molecule or a combination thereof. The dormancy factor may be selected from the group consisting of cytokines, chemokines, enzymes, extracellular vesicles, growth factors, and combinations thereof. The dormancy factor may be secreted by bone marrow niche cells (e.g., hFOBs) into the culture medium.

The term “reactivation factor” used herein refers to an agent soluble in a culture medium that maintains reactivation of tumor cells in the culture medium or promotes transition of tumor cells in the culture medium from a dormant state to a reactivation state. The agent may be a chemical compound, a biological molecule or a combination thereof. The reactivation factor may be selected from the group consisting of cytokines, chemokines, enzymes, extracellular vesicles, growth factors, and combinations thereof. The reactivation factor may be secreted by bone marrow niche cells (e.g., hMSCs) into the culture medium.

The term “predetermined period of time” used herein refers to at least 5, 7, 8, 9, 10, 11, 12, 13, 14, 15, 16, 17, 18, 19, 20, 21, 22, 23, 24, 25, 26 27 or 28 days, or at least 5, 6, 8, 9, 10, 11 or 12 weeks, or 10-25, 15-20, 10-20 or 15-25 days.

An indirect co-culture is provided. The indirect co-culture comprises bone marrow niche cells, tumor cells and a culture medium. The bone marrow niche cells and the tumor cells are incubated in the culture medium without direct contact between the bone marrow niche cells and the tumor cells. The bone marrow cells and the tumor cells are viable. At least 50%, 55%, 60%, 65%, 70%, 75%, 80%, 85%, 90%, 95% or 99% of the bone marrow niche cells may be viable for a predetermined period of time. At least 50%, 55%, 60%, 65%, 70%, 75%, 80%, 85%, 90%, 95% or 99% of the tumor cells may be viable for a predetermined period of time. In one embodiment, at least 65% of the tumor cells are viable for at least 15 days.

In the indirect co-culture, the tumor cells may be dormant. For example, at least 70%, 75%, 80%, 85%, 90%, 95% or 99% of the tumor cells may be dormant for at least 5, 7, 8, 9, 10, 11, 12, 13, 14, 15, 16, 17, 18, 19, 20, 21, 22, 23, 24, 25, 26 27 or 28 days. The bone marrow niche cells may be hFOBs. The culture medium may comprise a soluble dormancy factor. The dormancy factor may be secreted by the bone marrow niche cells, for example, hFOBs. The dormancy factor may be present in an amount effective for maintaining or promoting dormancy of the tumor cells.

In the indirect co-culture, the tumor cells may be reactivated. For example, at least 70%, 75%, 80%, 85%, 90%, 95% or 99% of the tumor cells may be reactivated for at least 5, 7, 8, 9, 10, 11, 12, 13, 14, 15, 16, 17, 18, 19, 20, 21, 22, 23, 24, 25, 26 27 or 28 days. The bone marrow niche cells may be hMSCs. The culture medium may comprise a soluble reactivation factor. The reactivation factor may be secreted by the bone marrow niche cells, for example, hMSCs. The reactivation factor may be present in an amount effective for maintaining or promoting reactivation of the tumor cells.

In the indirect co-culture, the culture medium may comprise an autophagy inhibitor. The autophagy inhibitor may be hydroxychloroquine (HCQ), other inhibitors of autophagosome/autophagolysosome formation, agents targeting autophagy signaling pathways or a combination thereof. The autophagy inhibitor may eliminate or reduce autophagy by at least 10%, 20%, 30%, 40%, 50%, 60%, 70%, 80%, 90% or 95% in the tumor cells.

In the indirect co-culture, the bone marrow niche cells may grow in a 3D matrix, which may be a hydrogel having Young's modulus (E) of 0.1-5.0 or 0.1-1.0 kPa, for example, 0.5 kPa. The tumor cells may grow in a 3D matrix, which may be a hydrogel having Young's modulus (E) of 0.1-5.0 or 0.1-1.0 kPa, for example, 0.5 kPa. The bone niche cells and the tumor cells do not grow in the same 3D matrix.

A method for preparing an indirect co-culture is provided. The preparation method comprises incubating bone marrow niche cells and tumor cells in a culture medium without direct contact between the bone marrow niche cells and the tumor cells. The bone marrow cells and the tumor cells are viable. At least 50%, 55%, 60%, 65%, 70%, 75%, 80%, 85%, 90%, 95% or 99% of the bone marrow niche cells may be viable for a predetermined period of time. At least 50%, 55%, 60%, 65%, 70%, 75%, 80%, 85%, 90%, 95% or 99% of the tumor cells may be viable for a predetermined period of time. In one embodiment, at least 65% of the tumor cells are viable for at least 15 days.

According to the preparation method of the present invention, the tumor cells may be dormant. For example, at least 70%, 75%, 80%, 85%, 90%, 95% or 99% of the tumor cells may be dormant for at least 5, 7, 8, 9, 10, 11, 12, 13, 14, 15, 16, 17, 18, 19, 20, 21, 22, 23, 24, 25, 26 27 or 28 days. The bone marrow niche cells may be hFOBs. The culture medium may comprise a soluble dormancy factor. The dormancy factor may be secreted by the bone marrow niche cells, for example, hFOBs. The dormancy factor may be present in an amount effective for maintaining or promoting dormancy of the tumor cells.

According to the preparation method of the present invention, the tumor cells may be reactivated. For example, at least 70%, 75%, 80%, 85%, 90%, 95% or 99% of the tumor cells may be reactivated for at least 5, 7, 8, 9, 10, 11, 12, 13, 14, 15, 16, 17, 18, 19, 20, 21, 22, 23, 24, 25, 26 27 or 28 days. The bone marrow niche cells may be hMSCs. The culture medium may comprise a soluble reactivation factor. The reactivation factor may be secreted by the bone marrow niche cells, for example, hMSCs. The reactivation factor may be present in an amount effective for maintaining or promoting reactivation of the tumor cells.

According to the preparation method of the present invention, the culture medium may comprise an autophagy inhibitor. The autophagy inhibitor may be hydroxychloroquine (HCQ), other inhibitors of autophagosome/autophagolysosome formation, agents targeting autophagy signaling pathways or a combination thereof. The autophagy inhibitor may eliminate or reduce autophagy by at least 10%, 20%, 30%, 40%, 50%, 60%, 70%, 80%, 90% or 95% in tumor cells in the culture medium.

According to the preparation method of the present invention, the bone marrow niche cells may grow in a 3D matrix, which may be a hydrogel having Young's modulus (E) of 0.1-5.0 or 0.1-1.0 kPa, for example, 0.5 kPa. The tumor cells may grow in a 3D matrix, which may be a hydrogel having Young's modulus (E) of 0.1-5.0 or 0.1-1.0 kPa, for example, 0.5 kPa. The bone niche cells and the tumor cells do not grow in the same 3D matrix.

For each preparation method of the present invention, an indirect co-culture prepared according to the method is provided.

A method for screening for an agent capable of inhibiting reactivation of dormant tumor cells is provided. This screening method comprises incubating bone marrow niche cells and dormant tumor cells in a culture medium without direct contact between the bone marrow niche cells and the dormant tumor cells such that the dormant tumor cells become reactivated. The screening method further comprises adding a test agent into the culture medium, and determining the percentage of the dormant tumor cells that become reactivated before and after the test agent is added. A decrease in the percentage of the reactivated tumor cells after the addition of the test agent indicates that the test agent inhibits reactivation of the dormant tumor cells. For example, where at least 5%, 10%, 20%, 30%, 40%, 50%, 60%, 70%, 80%, 90% or 95% fewer dormant tumor cells become reactivated for at least 5, 7, 8, 9, 10, 11, 12, 13, 14, 15, 16, 17, 18, 19, 20, 21, 22, 23, 24, 25, 26 27 or 28 days after the test agent is added into the culture medium, the test agent may be deemed to be capable of inhibiting reactivation of the dormant tumor cells. The bone marrow niche cells may be hFOBs. The test agent may be a chemical compound, a biological molecule or a combination thereof.

A method for screening for an agent capable of promoting dormancy of proliferating tumor cells is provided. The screening method comprises incubating bone marrow niche cells and proliferating tumor cells in a culture medium without direct contact between the bone marrow niche cells and the proliferating tumor cells such that the proliferating tumor cells become dormant. The screening method further comprises adding a test agent into the culture medium, and determining the percentage of the proliferating tumor cells that become dormant before and after the test agent is added. An increase in the percentage of the dormant tumor cells after the addition of the test agent indicates that the test agent promotes dormancy of the tumor cells. For example, where at least 5%, 10%, 20%, 30%, 40%, 50%, 60%, 70%, 80%, 90% or 95% fewer proliferating tumor cells become dormant for at least 5, 7, 8, 9, 10, 11, 12, 13, 14, 15, 16, 17, 18, 19, 20, 21, 22, 23, 24, 25, 26 27 or 28 days after the test agent is added into the culture medium, the test agent may be deemed to be capable of promoting dormancy of the proliferating tumor cells. The bone marrow niche cells may be hMSCs. The test agent may be a chemical compound, a biological molecule or a combination thereof. The screening method may further comprise inhibiting autophagy by, for example, adding an autophagy inhibitor. The autophagy inhibitor may be hydroxychloroquine.

### Example 1. Bio-Inspired Bone Marrow Metastatic Niche Microenvironment: In Vitro Well-Defined 3D Co-Culture Model for Cancer Dormancy and Reactivation

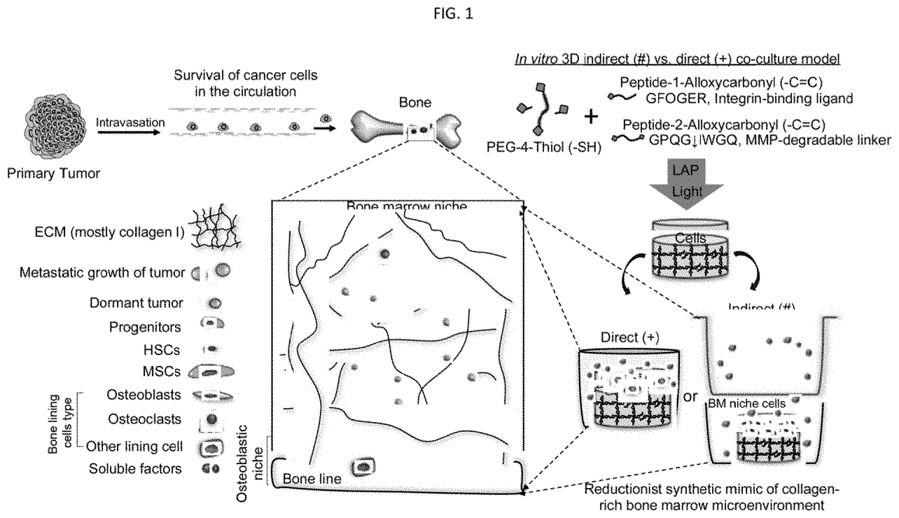

As shown in FIG. 1, a well-defined 3D microenvironment inspired by the in vivo bone marrow niche (collagen-rich, Young's modulus (E) ˜0.5 kPa) was created to allow examination of key secreted factors in long-term ER+ breast cancer cellular dormancy and reactivation induced by bone marrow niche cells. Specifically, a synthetic matrix comprised of crosslinked bioinert poly(ethylene glycol) (PEG) and bioactive peptides was used for the 3D co-culture of key bone marrow niche cells and breast cancer cells directly and indirectly. Importantly, the well-defined 3D indirect co-culture system provides control of cell-cell and cell-matrix interactions to enable investigations of specific secreted factors that regulate this complex process and therapeutic strategies for targeting them.

### Example 2. Viability and Metabolic Activity of ER+ BCCs in 3D Direct and Indirect Co-Culture Over Time

Bone marrow niche cells especially human mesenchymal stem cells (hMSCs) and human bone lining osteoblast cells (hFOBs) were observed to play a key role for insights into the mechanism of survival and reactivation of dormant ER+ breast cancer cells in these microenvironments. For example, BCCs are viable yet have limited growth in indirect co-culture with hFOBs and are viable and exhibited increased growth in presence of hMSC.

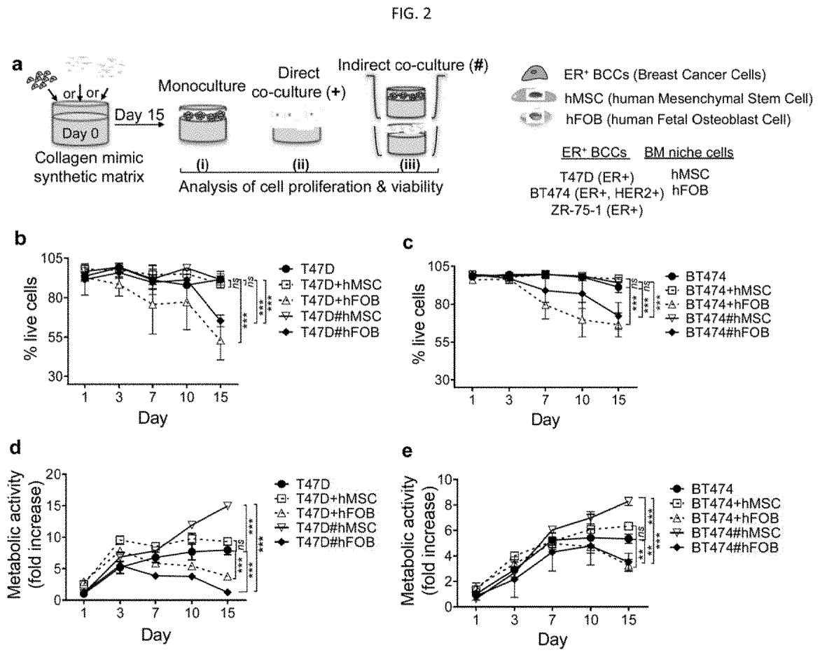

FIG. 2 demonstrates that BCCs are viable yet have limited growth in indirect co-culture with hFOBs, which creates a dormancy-promoting microenvironment, and are viable and exhibited increased growth in presence of hMSCs, which creates a growth-promoting microenvironment. a, Approach for direct and indirect 3D co-culture of bone marrow (BM) niche cells (e.g., hMSC or hFOB) with BCCs using synthetic based matrices, here in multiwell or transwell plates. b & c, ER+T47D or ER+ HER2+BT474 alone (mono-culture growth control), in direct co-culture (+), or in indirect co-culture (#)). Quantitative analysis of viable cells. d & e, Metabolic activity over time. Significant differences assessed by one-way analysis of variance (ANOVA) with Tukey's multiple comparisons test, where differences are shown for comparison between multiple conditions at day 15 time point (*p<0.05, **p<0.01; ***p<0.001; not significant (ns)). Similar data were obtained for ER+ZR-75-1. Data are shown represent mean±SD (n=3). Use of indirect co-culture approach allows elucidation of significant differences in responses of different BCCs and niche cells for clear observations of breast cancer dormancy or growth over long culture times.

### Example 3. Bone Marrow Niche Cell Secreted Factors

The model system established here allows facile probing effects of bone marrow niche cell secreted factors on dormancy (viable cells, not proliferating, capable of reproliferating upon stimulation): for example, co-cultures with hFOB cells were observed to induce dormancy of BCCs and hMSCs promote proliferation, and switching of these microenvironments allowed the elucidation of effects of transitioning from dormancy-promoting to growth-promoting microenvironments.

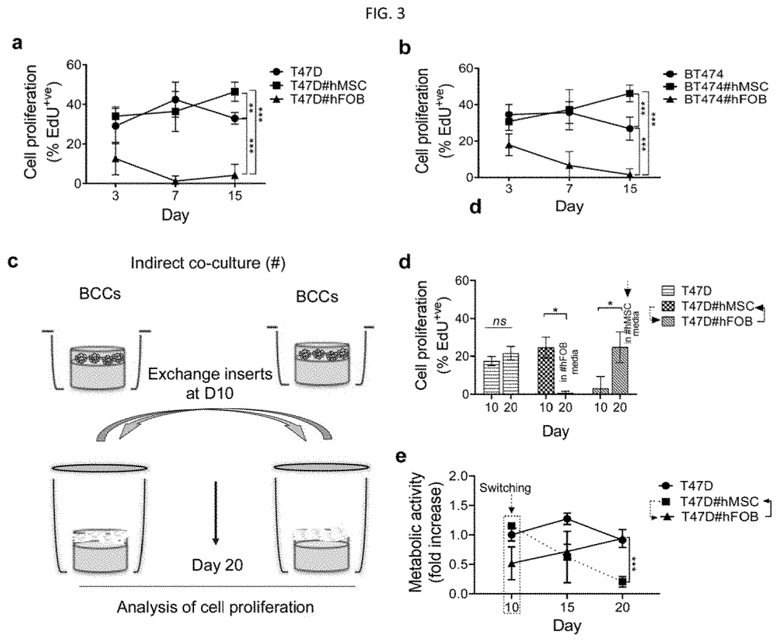

FIG. 3 shows probing effects of bone marrow niche cell secreted factors with indirect 3D co-cultures: hFOB cells induce dormancy of BCCs, whereas hMSC promote growth of dormant cells. Indirect co-culture also enables facile switching to elucidate effects of transition from dormancy-promoting to growth-promoting microenvironments. Breast cancer cell proliferation assessment of over time culture with different conditions in 3D hydrogels: 5-ethynyl-2′-deoxyuridine (EdU) proliferation assay was measured for both T47D & BT474 cells at each time point during 15 days of culture. a & b, Quantification of proliferative (EdU^{+ve}) T47D & BT474 cells alone (control) or in indirect co-culture (#) with different BM niche cells (hFOB or hMSC). Data indicate cells remained proliferative in control and co-culture with hMSC (T47D #hMSC & BT474#hMSC) cells, whereas a decrease in proliferation was observed for co-culture with hFOB (T47D #hFOB & BT474#hFOB) cells. c, Approach for assessing response to change in microenvironment with transwell insert exchange, here with different BM niche cells (hFOB or hMSC) at day 10. d, Proliferation assessment with insert exchange using EdU proliferation assay, here with quantitative analysis of EdU^{+ve} T47D cells before and after insert exchange. T47D cells from the co-cultured condition with hFOB exhibited an increase in the number of EdU^{+ve} cells when transferred into #hMSC (T47D #hFOB/hMSC) media at day 20, whereas T47D cells from the co-cultured condition with hMSC exhibited a decrease in the number of EdU^{+ve} cells when transferred into #hFOB (T47D #hMSC/hFOB) media. Significant differences assessed by Student's two-sided t-test, where differences shown for comparison between time points in mono and co-culture conditions (*p<0.05, **p<0.01; ***p<0.001). e, Quantitative analysis of metabolic activity with AlamarBlue assay of T47D cells after insert exchange at day 10 (fold change relative to day 3). Statistical differences determined with ANOVA with Tukey's multiple comparisons test, where differences are shown for comparison between co-culture conditions on day 15 and 20 (*p<0.05, **p<0.01; ***p<0.001). Data are shown represent mean±SD (n=3).

### Example 4. Autophagy in Dormant Tumor Cells

In vitro dormant ER+ BCCs survive through an autophagy mechanism in these bone marrow microenvironments. These observations in the model system are consistent with observations of ER+ breast cancer dormancy in bone in a preclinical animal model, where dormancy of the colonized ER+ breast cancer close to the bone lining site occurs through the survival and autophagy mechanisms.

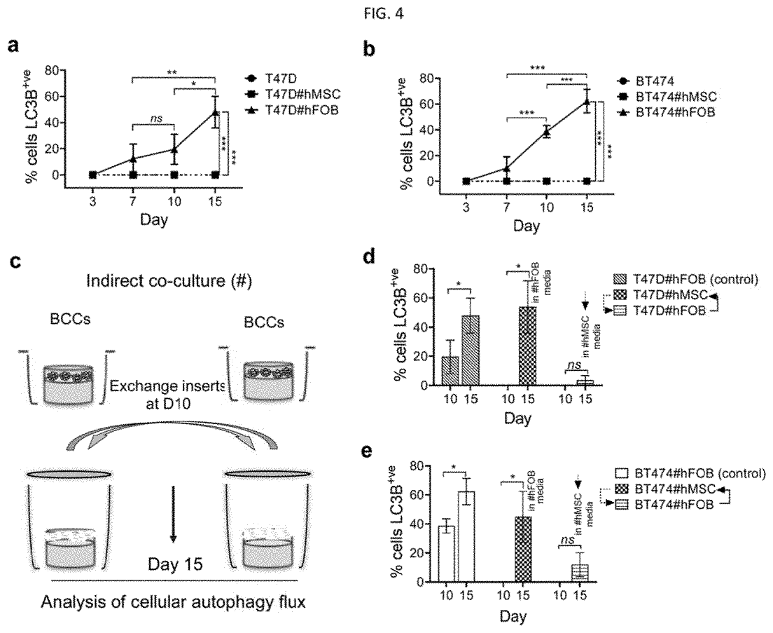

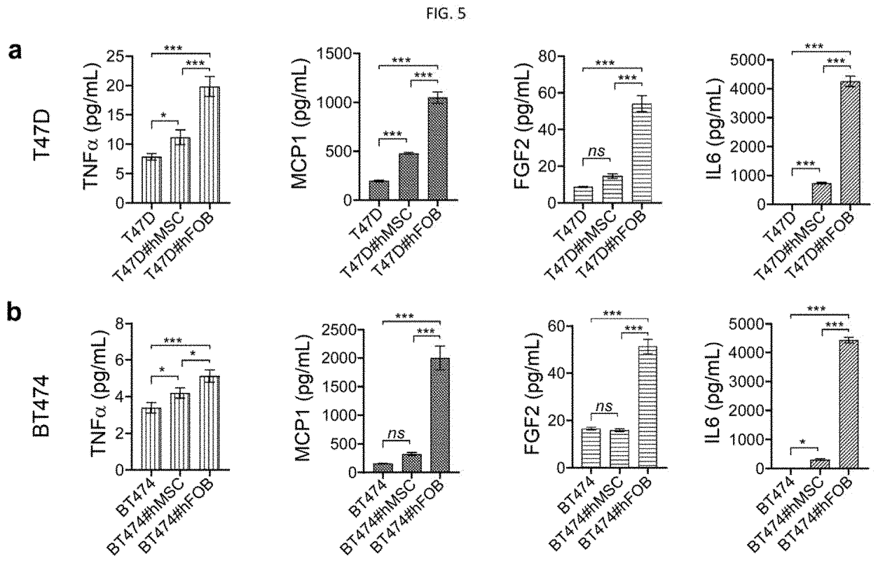

FIG. 4 shows elucidation of one mechanism of survival for dormant breast cancer cells within the model system: breast cancer cells exhibit induction of autophagy upon entering a dormant state in 3D indirect co-culture over time. Observations made with the in vitro model system shown here are consistent with in vivo observations in bone in a preclinical model for studying breast cancer dormancy at metastatic sites. a & b, Quantitative analysis of cells positive for LC3B^{+ve} puncta in images of immunofluorescent staining of autophagic protein (LC3B), cytoplasm (Phalloidin), and DNA (Hoechst) of T47D and BT474 cells in 3D mono-culture or indirect co-culture (#) with different BM niche cells (hFOB or hMSC) analyzed. Induction of autophagy observed in the indirect co-culture condition for T47D or BT474 with hFOB at Day 10 & 15, but not in mono-culture of T47D or co-culture of T47D with hMSC cells. Significant differences assessed by ANOVA with Tukey's multiple comparisons test, where differences are shown for comparison between multiple conditions at day 15 time point (*p<0.05, **p<0.01; ***p<0.001. c, Schematic representation of switching microenvironment by insert exchange between two different co-cultured conditions with T47D cells ((T47D #hMSC/hFOB) and (T47D #hFOB/hMSC)) at Day 10 and continued observation up to Day 15. d & e, Quantitative analysis of cells positive for LC3B^{+ve} puncta before and after transwell insert exchange. After exchanging of inserts at Day 10, T47D co-cultured with hFOB (T47D #hFOB) exhibited increased viability/proliferation and fewer LC3B^{+ve} puncta as compared to continual co-culture with #hFOB condition at Day 15. In the case of T47D #hMSC insert exchange to #hFOB, induction of autophagy was observed upon insert exchange with similar observations for BT474 cells in these co-cultures. Significant differences assessed by Student's two-sided t-test, where differences shown for comparison between time points in co-culture conditions (*p<0.05, **p<0.01; ***p<0.001). Data are shown represent mean±SD (n=3).

### Example 5. Factors Secreted by BM Niche Cells

Factors, including but not limited to cytokines and chemokines, secreted by the BM niche cells into indirect co-culture conditioned media can either induce dormancy or activation of ER+ breast cancer cells.

As shown in FIG. 5, a well-defined 3D indirect co-culture model allows facile assessment of factors secreted by cells for probing regulators of dormancy vs. persistent activation and reactivation. Here, the conditioned media was collected from 3D mono-culture and indirect (#) co-cultures of BM niche cells (hMSC & hFOB) with T47D or BT474 at day 10 and examined for specific cytokines with Luminex assay. The concentrations of secreted cytokines were normalized to fresh growth medium and expressed as pg/mL where averages are shown for a, T47D and b, BT474 cells in mono- or indirect co-culture. Significant differences assessed by ANOVA with Tukey's multiple comparisons test, where differences are shown for comparison between multiple conditions at day 10 time point (*p<0.05, **p<0.01; ***p<0.001). Data are shown represent mean±SD (n=3).

### Example 6. Cytokine Pathway in Dormant BCCs

Targeting of dormant ER+ breast cancer cells through the cytokine pathway can either improve survival or sustain the dormancy.

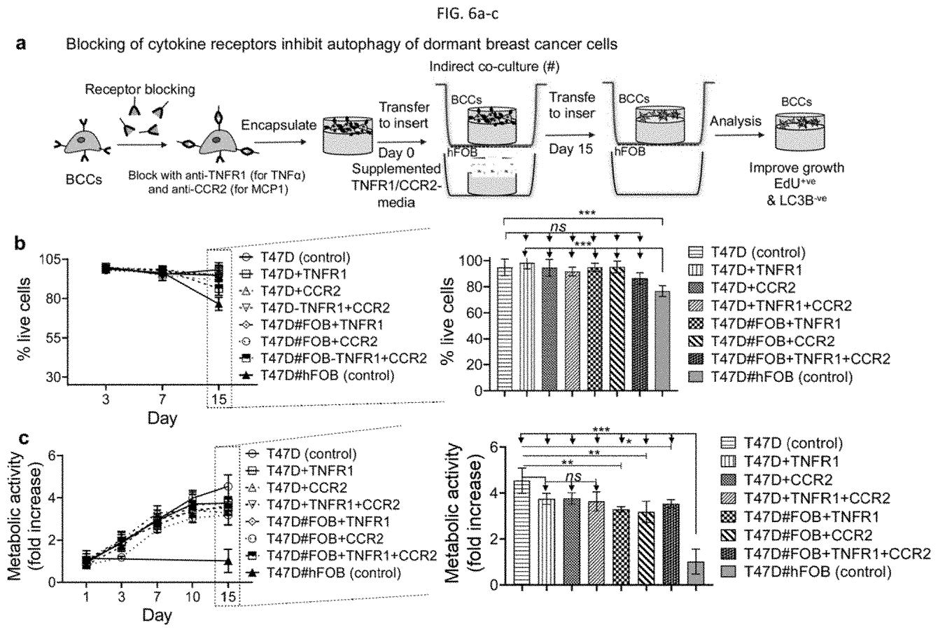

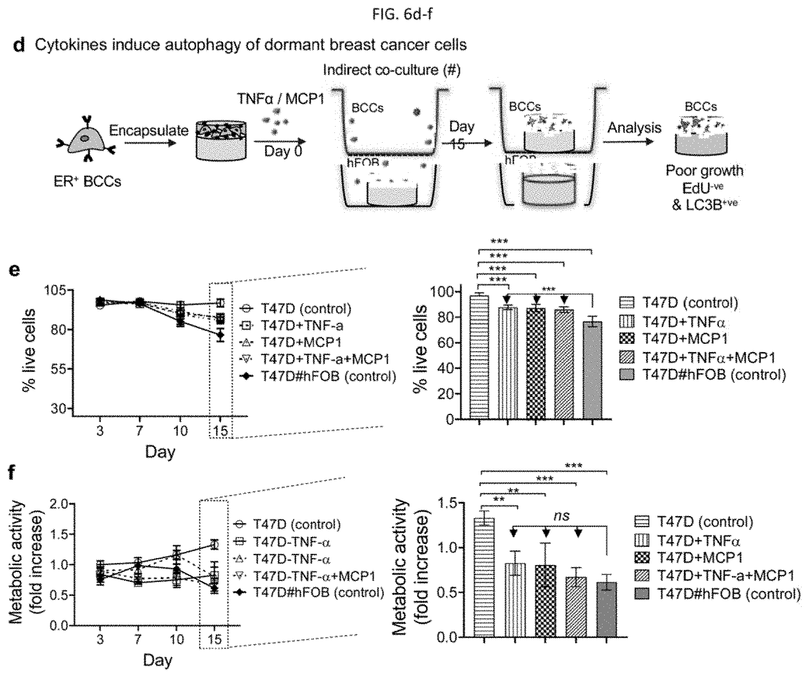

FIG. 6 shows probing effects of identified cytokines on BCCs viability and growth using well-defined 3D culture system to determine what factors are key in regulating dormancy and identify potential therapeutic strategies for targeting them. a, Approach for probing importance of cytokine binding on induction of dormancy and autophagy: BCCs were cultured with anti-TNFR1 (for TNFa) and anti-CCR2 (for MCP1) either in 3D mono-culture or indirect (#) co-culture with BM niche cells (hFOB). b, Quantitative analysis of viable BCCs in 3D culture (viability) after blocking of cytokine receptors for T47D either in mono-culture or in indirect co-culture (#) as compared to untreated controls of T47D cells or T47D cells co-cultured with hFOB (T47D #hFOB), where comparisons amongst conditions on day 15 are represented in the bar graph on the right. Treated cells exhibited improved growth in comparison to untreated dormant T47D co-cultured with hFOB cells. c, Metabolic activity over time for BCCs in 3D culture (untreated controls and treated T47D vs. indirect (#) T47D #hFOB co-culture) with quantitative comparison of conditions on day 15 to the right. d, Schematic representation of probing effects of cytokines (recombinant human TNFa and MCP1) on the induction of dormancy and autophagy in T47D cells in 3D cultures. e, Quantitative analysis of viable cells at each time point where day 15 comparisons are shown in the bar graph on the right. Treated conditions showed significant growth inhibition of T47D cells like untreated co-culture control (T47D #hFOB) and as compared to untreated control of T47D mono-culture. f, Metabolic activity at each time point where day 15 comparisons are shown in the bar graph on the right (untreated control or treated of T47D vs. indirect (#) T47D #hFOB co-culture). Significant differences were assessed by ANOVA with Tukey's multiple comparisons test, where differences are shown for comparison between controls and multiple conditions at day 15 time point (*p<0.05, **p<0.01; ***p<0.001). Data are shown represent mean±SD (n=3).

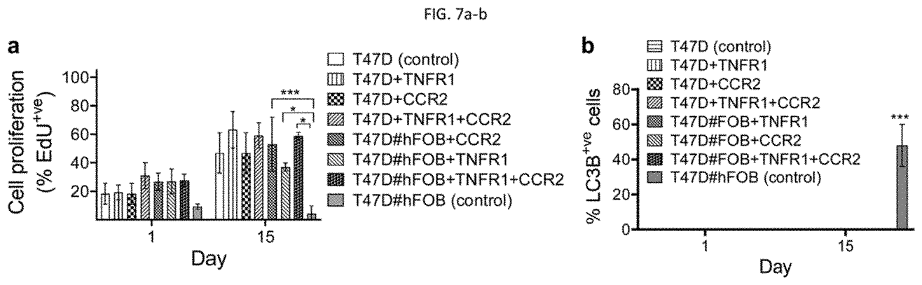

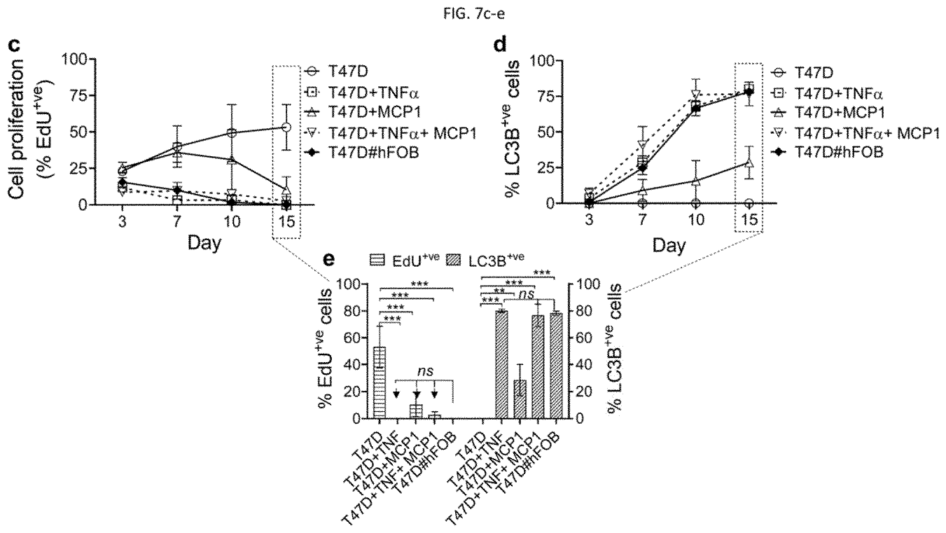

FIG. 7 shows probing effects of cytokines on induction of dormancy and autophagy: blocking of cytokine receptors (anti-TNFR1 and anti-CCR2) allowed proliferation of ER+ BCCs in dormancy-inducing hFOB co-culture, and treatment with recombinant human TNFa and MCP1 cytokines suppressed the growth of BCCs leading to the cellular dormancy and induction of autophagy. a, BCC proliferation over time in different 3D culture conditions with and without treatment with anti-TNFR1 and anti-CCR2 was assessed with EdU proliferation assay, quantifying EdU^{+ve} cells (here, T47D) at different time points in 3D culture (untreated controls or treated mono-culture and indirect co-cultures (#)). Observations indicated that cells remained proliferative in mono-culture control and in treated mono-culture and co-culture (T47D #hFOB) conditions, whereas a significant decrease in proliferation was observed for untreated co-culture control (T47D #hFOB). Results suggested that treatment with anti-TNFR1 and anti-CCR2 can maintain the proliferation rate and prevent dormancy of BCC in co-culture with osteoblast cells. b, Quantitative analysis of LC3B^{+ve} cells on day 1 and day 15 time points with and without treatment. Inhibition of autophagy was observed in the anti-cytokine receptor treatment co-culture conditions (after blocking of cytokine receptors using anti-TNFR1 and CCR2) in comparison to untreated co-culture control where formation of autophagosomes were observed in the dormant cells (LC3B^{+ve}, EdU^{+ve}). c & d, Induction of dormancy and autophagy in 3D culture with and without treatment with cytokines was assessed by quantitative analysis of EdU^{+ve} and LC3B^{+ve} puncta cells of T47D in comparison to controls (3D mono-culture or indirect co-culture with different BM niche cells (hFOB)). e, A bar graph of quantification analysis on EdU^{+ve} and LC3B^{+ve}T47D cells at day 15 time points from FIG. 7c & d. Results suggested that cytokine treatments significantly decreased the proliferation rate and induced autophagosome formation upon entering a dormant state in 3D culture over time, in comparison to the untreated T47D mono-culture. Also, treated cells showed similar induction of dormancy and autophagy (EdU⁻ve and LC3B^{+ve}) at day 15 like untreated co-culture dormancy control (T47D #hFOB) condition. Significant differences assessed by ANOVA with Tukey's multiple comparisons test, where differences are shown for comparison between controls and multiple conditions at day 15 time point (*p<0.05, **p<0.01; ***p<0.001). Data are shown represent mean±SD (n=3).

### Example 7. Regulation of Dormancy of BCCs

Inhibition of autophagy is beneficial in regulating the dormancy of ER+ breast cancer, where dosing strategy was observed to be important in the model system.

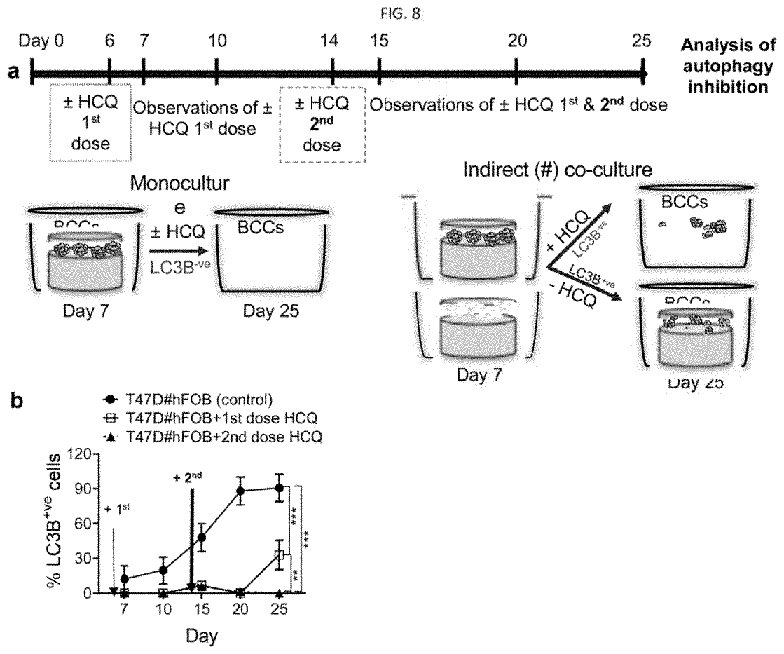

FIG. 8 shows that well-defined indirect 3D co-culture model allows evaluation of different therapeutic strategies for targeting dormancy survival mechanism: here, dose-dependent response to inhibitor of autophagy was examined. a, Overview of study for probing effects of inhibition of autophagy over time: 12.5 pg/mL hydroxychloroquine (HCQ) was used as a single dose on Day 6, and, for a subset of samples, a second dose was applied on Day 14 with continued observation over 25 days for T47D mono-culture and T47D #hFOB co-culture (control vs. treated conditions). b, Quantitative analysis of LC3B^{+ve} puncta cells in T47D #hFOB co-culture conditions with and without treatment. These observations indicated the inhibition of autophagy for cells in dormancy-inducing co-culture conditions with 2 doses of HCQ. Significant differences assessed by one-way ANOVA with Tukey's multiple comparisons test, where differences are shown for comparison between multiple time points (*p<0.05, **p<0.01; ***p<0.001). Data are shown represent mean±SD (n=3).

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

1. An indirect co-culture, comprising bone marrow niche cells, tumor cells and a culture medium, wherein the bone marrow niche cells and the tumor cells are incubated in the culture medium without direct contact between the bone marrow niche cells and the tumor cells, and wherein at least 65% of the tumor cells are viable for at least 15 days.

2. The indirect co-culture of claim 1, wherein at least 80% of the tumor cells are dormant.

3. The indirect co-culture of claim 2, wherein the bone marrow niche cells are human bone lining osteoblast cells (hFOBs).

4. The indirect co-culture of claim 2 or 3, wherein the culture medium comprises a soluble dormancy factor in an amount effective for promoting dormancy of the tumor cells.

5. The indirect co-culture of claim 4, wherein the soluble dormancy factor is selected from the group consisting of cytokines, chemokines, enzymes, extracellular vesicles, growth factors, and combinations thereof.

6. The indirect co-culture of claim 4, wherein the soluble dormancy factor is secreted by the bone marrow niche cells.

7. The indirect co-culture of claim 1, wherein at least 80% of the tumor cells are reactivated.

8. The indirect co-culture of claim 7, wherein the bone marrow niche cells are human mesenchymal stem cells (hMSCs).

9. The indirect co-culture of claim 7, wherein the culture medium comprises a soluble reactivation factor in an amount effective for promoting reactivation of the tumor cells.

10. The indirect co-culture of claim 9, wherein the soluble reactivation factor is selected from the group consisting of cytokines, chemokines, enzymes, extracellular vesicles, growth factors, and combinations thereof.

11. The indirect co-culture of claim 9, wherein the soluble reactivation factor is secreted by the bone marrow niche cells.

12. The indirect co-culture of claim 1, wherein the culture medium comprises an autophagy inhibitor.

13. The indirect co-culture of claim 12, wherein the autophagy inhibitor is hydroxychloroquine (HCQ).

14. The indirect co-culture of claim 1, wherein the tumor cells are selected from the group consisting of breast cancer cells, bone cancer cells, brain cancer cells, lung cancer cells, prostate cancer cells, and skin cancer cells.

15. The indirect co-culture of claim 1, wherein the tumor cells are estrogen receptor-positive (ER+) breast cancer cells.

16. The indirect co-culture of claim 1, wherein the tumor cells are obtained from a patient having an associated primary tumor.

17-20. (canceled)

21. A method for preparing an indirect co-culture, comprising incubating bone marrow niche cells and tumor cells in a culture medium without direct contact between the bone marrow niche cells and the tumor cells, wherein at least 65% of the tumor cells are viable for at least 15 days.

22-40. (canceled)

41. An indirect co-culture prepared according to the method of claim 21.

42. A method for screening for an agent capable of inhibiting reactivation of dormant tumor cells in the indirect co-culture of claim 1, comprising:
(a) incubating bone marrow niche cells and dormant tumor cells in a culture medium without direct contact between the bone marrow niche cells and the dormant tumor cells, whereby at least 80% of the dormant tumor cells become reactivated;
(b) adding a test agent into the culture medium, and
(c) determining the percentage of the dormant tumor cells that become reactivated before and after the test agent is added, wherein a decrease in the percentage of the reactivated tumor cells after the addition of the test agent indicates that the test agent inhibits reactivation of the dormant tumor cells.

43. (canceled)

44. A method for screening for an agent capable of promoting dormancy of proliferating tumor cells in the indirect co-culture of claim 1, comprising:
(a) incubating bone marrow niche cells and proliferating tumor cells in a culture medium without direct contact between the bone marrow niche cells and the proliferating tumor cells, whereby at least 80% of the proliferating tumor cells become dormant;
(b) adding a test agent into the culture medium, and
(c) determining the percentage of the proliferating tumor cells that become dormant before and after the test agent is added, wherein an increase in the percentage of the dormant tumor cells after the addition of the test agent indicates that the test agent promotes dormancy of the tumor cells.

45. (canceled)

46. (canceled)
